(19) United States
(12) Patent Application Publication
(10) Pub. No.: US 2013/0017197 A1
(43) Pub. Date: Jan. 17, 2013

(54) STABILIZED ANTIBODY PREPARATIONS AND USES THEREOF

(71) Applicants: Robert Gurny, Geneva (CH); Leonardo Scapozza, Grens (CH); Yvonne Westermaier, Flims (CH); Marieke Veurink, Geneve (CH)

(72) Inventors: Robert Gurny, Geneva (CH); Leonardo Scapozza, Grens (CH); Yvonne Westermaier, Flims (CH); Marieke Veurink, Geneve (CH)

(73) Assignee: UNIVERSITE DE GENEVE, GENEVA 4 (CH)

(21) Appl. No.: 13/637,041

(22) PCT Filed: Mar. 31, 2011
(86) PCT No.: PCT/IB11/51372

§ 371 Date: Sep. 25, 2012

Related U.S. Application Data
(60) Provisional application No. 61/319,310, filed on Mar. 31, 2010.

(51) Int. Cl. A61K 39/395; A61P 29/00; A61P 37/06; A61P 7/02; A61P 27/02; A61P 1/00; A61P 9/00; A61P 37/00; A61P 11/06; A61P 17/06; A61P 35/00; A61P 31/12

(52) U.S. Cl. 424/133.1; 424/130.1; 424/178.1

(57) ABSTRACT

The present invention is directed to stabilized intact antibody formulations, related methods and uses thereof. In particular, the invention relates to a method of stabilizing an intact antibody in a liquid carrier.

### FIELD OF THE INVENTION

The present invention relates to antibody preparations, in particular to antibody preparations having increased stability, and the uses thereof. The invention further relates to pharmaceutical compositions comprising a stabilized antibody preparation and methods for stabilizing antibody preparations.

### BACKGROUND OF THE INVENTION

Therapeutic antibodies are currently the fastest growing area of biopharmaceuticals. The recent development of chimeric and fully-humanized monoclonal antibodies has spawned an unprecedented interest in using these molecules as therapeutic agents since they can specifically target molecules implicated in disease, thus essentially side-stepping the secondary effects that may be associated with conventional drug therapies. Recent progress in gene recombinant technology has enabled the large scale production of physiologically active proteins such as monoclonal antibodies for diagnostic and therapeutic applications.

The provision of stable therapeutic protein formulations, in particular stable antibody formulations, presents a challenge. Physical and chemical instability of antibodies in aqueous media is a complex function of solution conditions and temperature. Antibodies are, for example, susceptible to deamidation, isomerization, oxidation, proteolysis, aggregation and other covalent modifications. Degradation of antibody formulations due to aggregation phenomena is a particular problem. Not only does the formation of aggregates lead to a reduction in antibody activity, thereby reducing the efficacy of the protein drug, but may also result in potential clinical side-effects or toxicity since aggregates can increase the immunogenicity of the protein drug (Demeule et al., 2006, Eur. J. Pharm. Biopharm., 62:121-30; Sauerborn et al., 2009, Cell Press., 53-58).

Antibody aggregation is also a source of batch to batch variations in the antibody production chain and its control leads to regulatory and quality control burdens, with their associated costs.

Further, the propensity of antibodies to aggregate adversely affects the stability of therapeutic antibody formulations on storage, including their shelf-life, and their useable administration time once removed from optimal storage conditions.

Unlike other model proteins, antibody stability is not necessarily dependent on protein concentration, buffer concentration, salt concentration, or agitation. Antibody stabilization is problematic since antibodies are very sensitive to environmental conditions which render aggregation and degradation very difficult to predict, notably because each individual antibody may have a very specific and characteristic stability profile. The lack of effect for primary factors commonly known to affect physical stability suggests that the mechanism(s) of antibody stability is/are counter-intuitive and may differ from other well-studied proteins.

To date, most therapeutic monoclonal antibodies introduced into clinical use are of the antibody type immunoglobulin G (IgG). For example, bevacizumab (Avastin®) is a recombinant monoclonal humanized IgG1 antibody with a molecular weight of 149 kDa that binds to and inhibits the biologic activity of vascular endothelial growth factor (VEGF). VEGF is known to play a pivotal role in tumor angiogenesis and is a significant mitogenic stimulus for arterial, venous and lymphatic endothelial cells. The addition of bevacizumab to chemotherapy has been shown to increase overall response rate, duration of response and survival for patients with metastatic colon cancer. Bevacizumab is beneficial in first line non-small cell lung cancer, metastatic breast cancer and second line metastatic colorectal cancer. Bevacizumab is also beneficial in the treatment of neovascular age-related macular degeneration (AMD), a common form of progressive age-related vision loss.

A number of approaches have been investigated to attempt to improve antibody stability. These include approaches based on the addition of ‘stabilizing’ agents to a solution containing the immunoglobulin, and attempts to engender single amino acid mutations at the site(s) implicated in the formation of aggregates on the immunoglobulin molecules. Examples of species investigated as ‘stabilizing’ agents in prior attempts to improve stability of immunoglobulin in solution include polysorbate-based surfactants (GB 2175906), amino acids (EP 0025275, WO 2005/049078), polyethers (EP 0018609), glycerin, albumin, dextran sulphate (U.S. Pat. No. 4,808,705). The success of this approach has, however, been limited. It is believed that one reason for this limited success is that the ‘stabilizing’ agents are directed at optimizing the environment in which the immunoglobulin is contained, not specifically at interfering with the mechanism of interaction of immunoglobulin molecules in the formation of aggregates. This approach also has limitations in regard of the quantity of stabilizing agent(s) that may be required to exert a positive effect; such quantities may have other detrimental effects on immunoglobulin molecules such as protein unfolding (e.g. for surfactants), or on the suitability and safety of the ‘stabilized’ preparations for subsequent clinical administration.

Single amino acid mutations to immunoglobulins could provide a method of specifically targeting sites implicated in aggregation, but such an approach obviously modifies the structure of the immunoglobulin, and this may affect both its clinical efficacy, and its immunogenicity in the recipient which can create undesirable side effects such as an immune response against the therapeutic agent.

Although many different approaches have been proposed, and some methods have been incorporated into antibody formulations, aggregation is still an issue. There is to date no available single, effective and widely applicable solution to the aggregation of immunoglobulins used for clinical applications.

Since aggregation is a major issue for the production, formulation and stability of therapeutic antibodies, and can lead to loss of biological activity, loss of solubility and even increased immunogenicity, there is an ongoing need to provide therapeutic antibody preparations, particularly formulations of monoclonal antibodies, which provide improved shelf-life and stability of those antibodies.

### SUMMARY OF THE INVENTION

It has been unexpectedly found by the inventors that liquid preparations of intact antibodies, in particular intact monoclonal antibodies, may be effectively stabilized by the addition of a compound of formula (I) according to the invention. It has further been surprisingly found by the inventors that compounds of formula (I) according to the invention provide stabilizing effects on liquid preparations of intact antibodies even when present at very low concentrations.

According to one aspect of the invention, there is now provided a stable antibody formulation comprising a liquid carrier, an intact antibody and a compound of the formula (I):

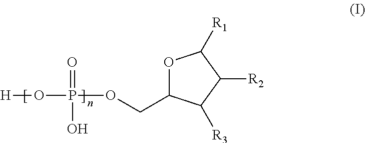

wherein R₁ is a nucleobase; R₂ is H or OR₄ wherein R₄ is H or a C ₁₋₄ alkyl group; R₃ is H or OR₅ wherein R₅ is H or a C₁₋₄ alkyl group; and n is an integral from 1-3 (i.e. selected from 1, 2 and 3), or a pharmaceutically acceptable salt or a tautomer thereof. The nucleobase R₁ may be selected from the group comprising adenine, guanine, thymine, uracil, xanthine, ethanoadenine, inosine, orotidine, or cytosine.

Compounds of the formula (I) have been shown to reduce the propensity of intact antibodies, such as, for example, the intact monoclonal antibody bevacizumab, to form aggregates in liquid formulations. Compounds of the formula (I) have been shown to induce the reversion, or breaking, of already formed aggregates of intact antibodies, such as for example bevacizumab, into an essentially monomeric state.

Advantageously, stabilized formulations of intact antibodies, such as bevacizumab, according to the invention have been shown to have a decreased propensity to aggregate compared to known formulations.

The compound of formula (I) may be in the form of its free acid, or may be in the form of a pharmaceutically acceptable salt, for example in the form a sodium salt, e.g. a mono- or di-sodium salt.

According to one embodiment, R₂ is H and R₃ is OH. According to another embodiment R₂ and R₃ are both OH.

According to one embodiment, the compound of formula (I) is selected from the group comprising adenosine 5′-monophosphate (AMP), adenosine 5′-diphosphate (ADP), or adenosine 5′-triphosphate (ATP).

According to one embodiment, the compound of formula (I) is adenosine 5′-triphosphate (ATP) or a tautomer thereof.

According to one embodiment, the compound of formula (I) is guanosine 5′-monophosphate (GMP) or a tautomer thereof.

According to another embodiment, the compound of formula (I) is guanosine 5′-monophosphate (GMP).

According to one embodiment, the compound of formula (I) is adenosine 5′-monophosphate (AMP) or a tautomer thereof.

According to a preferred embodiment, the compound of formula (I) is adenosine 5′-monophosphate (AMP).

According to another aspect of the invention, there is provided a pharmaceutical formulation such as a formulation formulated for administration to a mammal (e.g. human) comprising a stable antibody formulation according to the invention or a stabilized antibody according to the invention.

According to another aspect of the invention, there is provided a pharmaceutical unit dosage form suitable for administration to a mammal comprising a pharmaceutical formulation according to the invention.

According to another aspect of the invention, there is provided a kit comprising, in one or more container(s), a formulation according to the invention together with instructions of use of said formulation.

According to another aspect of the invention, there is provided a formulation according the invention for use as a medicament.

In particular embodiments, the medicament may be for use in the treatment or prevention of a disease or disorder selected from immunological diseases, autoimmune diseases, infectious diseases, inflammatory diseases, neurological diseases, neovascular diseases, or oncological diseases.

According to embodiments of the invention, there is provided a formulation according the invention for the prevention or treatment of a disease or a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD).

According to another aspect of the invention, there is provided a method of stabilizing an intact antibody in aqueous solution according to the invention.

According to another aspect of the invention, there is provided a process for the preparation of a formulation of an intact antibody in aqueous solution according to the invention.

According to another aspect of the invention, there is provided a stabilized intact antibody or a formulation thereof obtainable by a process or a method according to the invention.

According to another aspect of the invention, there is provided a method of preventing, treating or ameliorating a disease or a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD), said method comprising administering in a subject in need thereof a prophylactic or therapeutically effective amount of a formulation according to the invention or of a stabilized intact antibody according to the invention.

According to another aspect of the invention there is provided a use of a formulation according to the invention or of a stabilized intact antibody according to the invention for the preparation of a pharmaceutical formulation for the prevention and/or treatment of a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD).

According to another aspect of the invention, there is provided a use of a formulation according to the invention or of a stabilized intact antibody according to the invention for inhibiting aggregation in the culture, preparation, purification and processing of antibodies prior to formulation into therapeutic preparations.

Other objects and advantages of the present invention will be apparent from the claims and the following detailed description, examples and accompanying drawings, wherein

FIG. 1 is a graphical representation of the stabilizing effect of the compound adenosine 5′-monophosphate on the monoclonal antibody bevacizumab formulated in an aqueous carrier, according to one embodiment of the invention as described in Example 1.

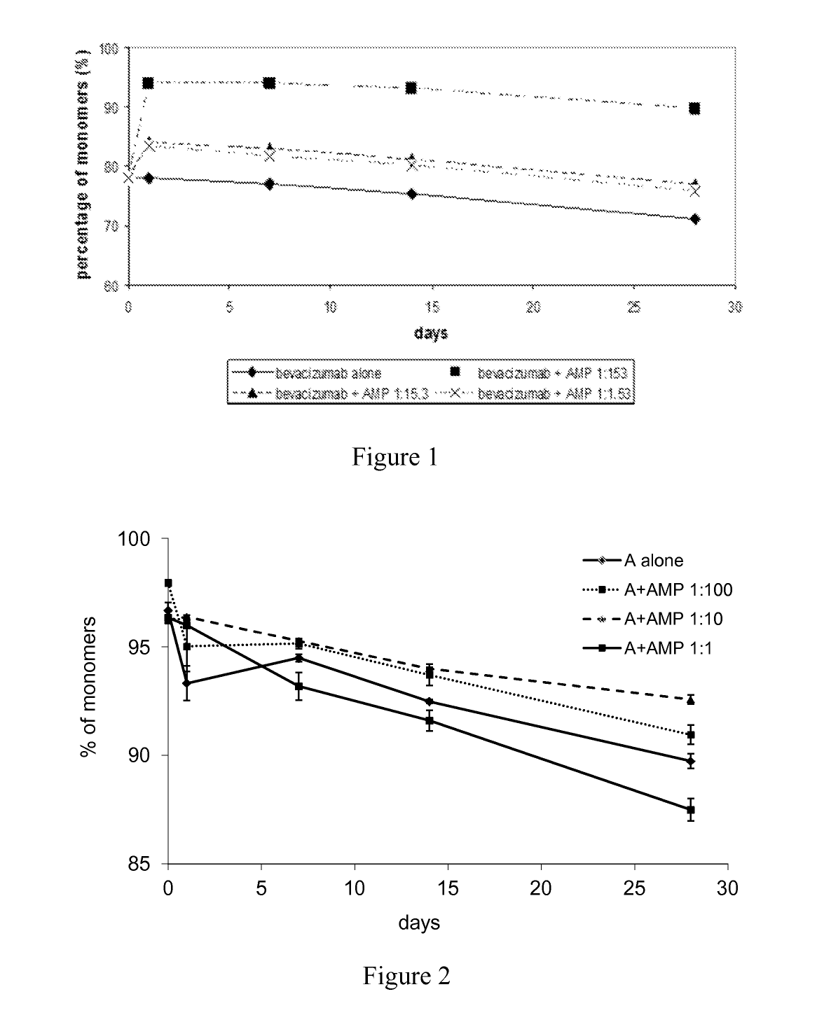

FIG. 2 is a graphical representation of the stabilizing effect of the compound adenosine 5′-monophosphate on a monoclonal antibody bevacizumab formulated in an unmodified commercial formulation (Avastin®, “A”) at different molar ratios as described in Example 2.

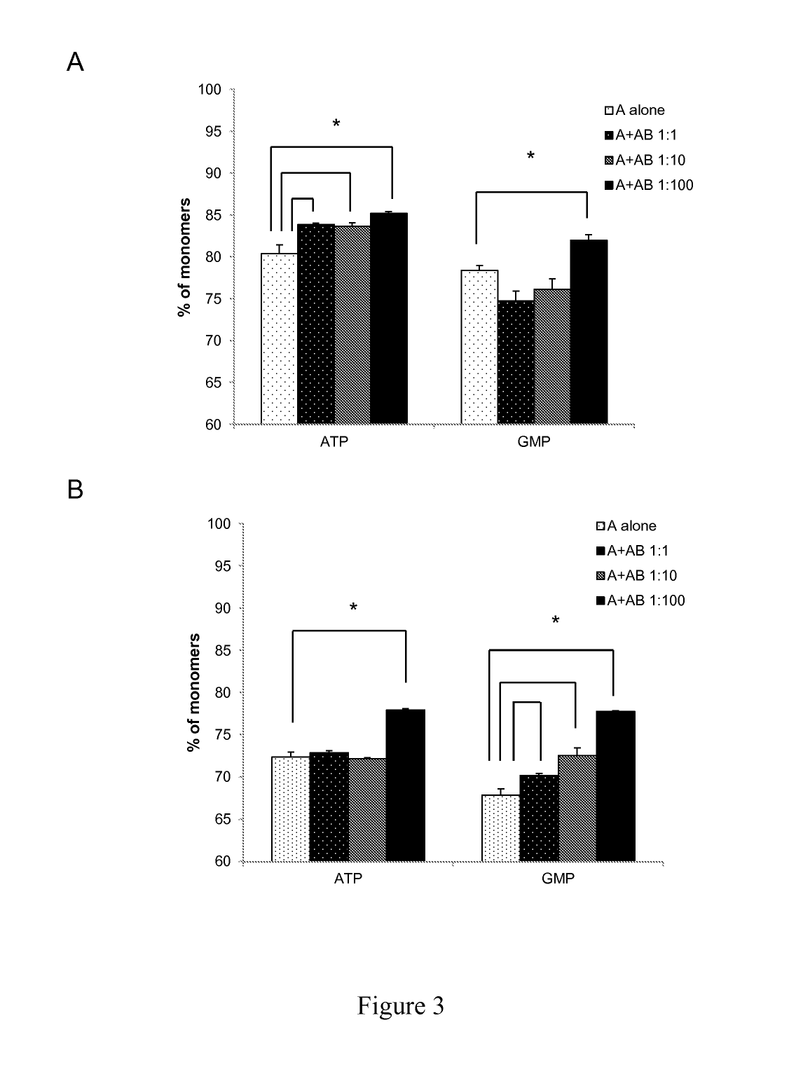

FIG. 3 represents Avastin® “A” stability comparison in presence and absence of a compound of formula (I) (ATP or GMP, “AB”) after storage at 40° C. as described in Example 3. A: after 1 day of storage (t₁); B: After 28 days of storage (t₂₈).The percentage of monomers is presented as mean (n=3)±SD. A significant increase in monomers for a combined formulation compared to Avastin® alone is represented by * (p<0.05).

### DETAILED DESCRIPTION OF THE INVENTION

The term “intact antibody”, as used herein, refers to antibodies which possess both Fab and Fc regions, as opposed to antibody fragments e.g. Fab, Fab1 or Fab2 fragments, or single chains thereof. Intact antibodies according to the invention present an aggregation propensity.

The term “monoclonal antibody”, as used herein, refers to a preparation of antibody molecules derived from a single clone of antibody producing cells of a uniform amino acid composition. A monoclonal antibody typically exhibits a binding specificity and affinity for a single epitope. Methods for the preparation of monoclonal antibodies are well-known in the art, and are widely based on hybridoma cell production techniques or recombinant antibody engineering techniques.

In embodiments of the invention, the intact antibody can be a full immunoglobulin molecule, particularly monomeric immunoglobulins, e.g. IgDs, IgEs and IgGs, such as IgG1, IgG2, IgG2b, IgG3 or IgG4.

In embodiments of the invention, the intact antibody can be a native antibody.

In other embodiments of the invention intact antibody can be an intact monoclonal antibody conjugated to an accessory molecule, also referred to herein as a “conjugated antibody”.

The term “accessory molecule” includes a molecule or an assembly of molecules, of natural or synthetic origin, attached or conjugated to the antibody molecule, providing additional therapeutic, diagnostic, analytical capability or imaging functionality, whereby such functionality is targeted, delivered or activated by the specificity of the antibody.

The accessory molecule may be, for example, an agent active for the treatment of cancer, such as a chemotherapeutic agent, or a radioactive agent.

In embodiments of the invention the intact antibody can be selected from known therapeutic, diagnostic or preventative intact monoclonal antibody drugs. For example, Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab or Ustekinumab, may be mentioned.

In a particular embodiment, an intact antibody according to the invention is bevacizumab, notably Avastin® such as described in Presta et al., Cancer Res., 57 (1997), 4593-4599.

The term “cancer” includes metastatic and non-metastatic cancers such as colon cancer, rectal cancer, breast cancer, renal cell carcinoma, glioblastoma multiforme, lung cancer, ovarian cancer, prostate cancer, liver cancer, pancreatic cancer, bone cancer, bone metastasis, leukemias, brain cancers, testicular cancer, uterine cancers, cervical cancers, endometrial cancer or other cancers responsive to monoclonal antibody-based therapy.

The term “age-related macular degeneration” (AMD) includes an eye progressive disease presenting an onset usually after age 60 that progressively destroys the macula, the central portion of the retina, impairing central vision.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject, which may be predisposed to the disease, but has not yet been diagnosed as having it, such as a preventive early asymptomatic intervention; (b) inhibiting the disease, i.e., arresting its development; or relieving the disease, i.e., causing regression of the disease and/or its symptoms or conditions such as improvement or remediation of damage. In particular, the methods, uses, formulations and compositions according to the invention are useful in the preservation of vision and/or prevention of vision loss in patients with age-related macular degeneration and/or in the treatment of cancers.

The term “subject” as used herein refers to mammals. For example, mammals contemplated by the present invention include humans, primates, domesticated animals such as cattle, sheep, pigs, horses, laboratory rodents and the like.

The term “effective amount” as used herein refers to an amount of at least one polypeptide or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

The term “efficacy” of a treatment according to the invention can be measured based on changes in the course of a disease in response to a use or a method according to the invention. For example, the efficacy of a treatment of a cancer according to the invention can be measured by a reduction of tumor volume, and/or an increase of progression free survival time.

The term “pharmaceutical formulation” refers to preparations which are in such a form as to permit biological activity of the active ingredient(s) to be unequivocally effective and which contain no additional component(s) which would be toxic to subjects to which the said formulation would be administered.

The term “pharmaceutically acceptable salt” refers to a salt that retains the desired activity of the defined compound (i.e. compound of formula (I)) and does not cause any undesired toxicological effects. According to certain embodiments of the invention the pharmaceutically acceptable salt may be a basic addition salt, such as a sodium, potassium, magnesium or calcium salt. A preferred pharmaceutically acceptable salt of a compound of formula (I) is a sodium salt, e.g. a mono- or di-sodium salt. The invention further encompasses any tautomers of the compounds according to the invention.

The term “stable” or “stabilized” refers in the context of the invention to formulations in which the antibody therein retains its physical stability (e.g. level of aggregation or aggregation propensity decreased, absence of precipitation or denaturation) and/or chemical stability (e.g. absence of chemically altered forms) upon storage or processing. Stability of the antibody formulations according to the invention may be measured by various techniques known to the skilled person in the art. For example, stability can be measured by aggregation state measurements (e.g. by Multi-Angle Light Scattering (MALS) after separation by Asymmetrical Flow Field-Flow Fractionation (AFFF), high performance size exclusion chromatography, analytical ultracentrifugation, fluorescence microscopy or electron microscopy). Preferably, the stability of the formulation is measured at a selected temperature and/or for a selected storage time. Typically, the stability of a formulation according to the invention is measured at a temperature of 40° C. for a period of 35 days. According to a particular embodiment, the stability of a formulation according to the invention is measured at a temperature of 40° C. for a period of at least 28 days.

According to one aspect of the invention there is provided a stable antibody formulation comprising an aqueous carrier, an intact antibody and a compound of the formula (I):

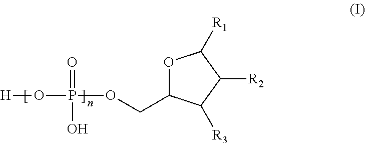

wherein R₁ is a nucleobase; R₂ is H or OR₄ wherein R₄ is H or a C₁₋₄ alkyl group; R₃ is H or OR₅ wherein R₅ is H or a C₁₋₄ alkyl group; and n is an integral from 1-3, or a pharmaceutically acceptable salt or tautomer thereof.

The term “alkyl” when used alone or in combination with other terms, comprises a straight chain or branched C₁-C₄ alkyl which refers to monovalent alkyl groups having 1 to 4 carbon atoms. This term is exemplified by groups such as methyl, ethyl, n-propyl, i-propyl, n-butyl, s-butyl, i-butyl, t-butyl and the like.

The nucleobase R₁ may be selected from the group comprising adenine, guanine, thymine, uracil, xanthine, ethanoadenine, inosine, orotidine, or cytosine. According to a preferred embodiment, the nucleobase is adenine. According to another embodiment, the nucleobase is guanine

The compound of formula (I) may be in the form of its free acid, or may be in the form of a pharmaceutically acceptable salt, for example in the form of a sodium salt, e.g. a mono- or di-sodium salt.

According to one embodiment, R₂ and R₃ are each independently H or OH. According to another embodiment, R₂ is H and R₃ is OH. According to another preferred embodiment, R₂ and R₃ are both OH.

According to one embodiment, a compound according to the invention is according to formula (I) wherein n is 1 and R₁, R₂ and R₃ are as defined in the present description.

According to one embodiment, a compound according to the invention is according to formula (I) wherein n is 3 and R₁, R₂ and R₃ are as defined in the present description.

Formulations according to the invention may contain one or more compound(s) of formula (I), or pharmaceutically acceptable salt(s) thereof.

Advantageously liquid preparations of intact antibodies, in particular intact monoclonal antibodies, may be effectively stabilized by the addition of a compound of formula (I) according to the invention.

Compounds of the formula (I) have been shown to advantageously reduce the propensity of intact antibodies, such as, for example, the intact monoclonal antibody bevacizumab, to form aggregates in liquid formulations.

Formulations, in particular aqueous formulations, of intact antibodies containing a compound of formula (I) according to the invention may exhibit, for example between 10 to 80%, e.g. between 30% to 70%, lower proportion of antibody in aggregate form after storage under accelerated storage conditions (e.g. at storage at a temperature of 40° C.) for between 1 to 30 days, compared to a corresponding formulation of the intact antibody not containing the compound of formula (I).

The present invention allows the preparation of formulations of intact antibody in aqueous carrier wherein less than 20%, even less than 15%, even less than 10% of the antibody is in aggregate form, as determined by MALS coupled to AFFF, during storage at a temperature of 40° C. for 35 days.

According to one embodiment, the invention provides a formulation according to the invention wherein less than 10% of bevacizumab is in aggregated form as determined by MALS coupled to AFFF during storage at a temperature of 40° C. for 35 days. Compounds of the formula (I) have been shown to advantageously induce the reversion, or breaking, of already formed aggregates of intact antibodies, such as for example bevacizumab, into an essentially monomeric state.

For example, the addition of a compound of formula (I) to a formulation, in particular an aqueous formulation, of intact antibodies containing already formed aggregates, for instance in which a proportion of at least 20% of the antibody molecules in the formulation are in aggregate form, makes it possible to induce the reversion of a significant proportion of the formed aggregates into an essentially monomeric state. For instance, an increase in the amount of antibody monomers in the formulation of, for example, from 5% to 50%, e.g. from 10% to 30%, may be exhibited, after addition of a compound of formula (I) according to the invention.

Further, advantageously, compounds of formula (I) according to the invention can provide stabilizing effects on liquid preparations of intact antibodies even when present at very low concentrations.

Advantageously stabilized formulations of intact antibodies, such as bevacizumab, according to the invention have been shown to have a decreased propensity to aggregate compared to known formulations.

Particular compounds according to formula (I) include: adenosine 5′-mono-, -di-, or -triphosphate, guanosine 5′-mono-, -di-, or -tri-phosphate, uridine 5′-mono-, -di-, or -tri-phosphate; cytidine 5′-mono-, -di-, or -triphosphate, deoxyadenosine 5′-mono-, -di-, or -triphosphate, deoxyguanosine 5′-mono-, -di-, or -triphosphate, thymidine 5′-mono-, -di-, or -triphosphate, deoxyuridine 5′-mono-, -di-, or -triphosphate, deoxycytidine 5′-mono-, -di-, or -triphosphate, xanthine 5′-mono-, -di-, or -triphosphate, ethoadenosine 5′-mono, -di-, or -triphosphate, inosine 5′-mono-, -di-, or -triphosphate, orotidine 5′-mono-, -di-, or -triphosphate.

According to one embodiment, the compound of formula (I) is selected from the group comprising adenosine 5′-monophosphate (AMP), adenosine 5′-diphosphate (ADP), or adenosine 5′-triphosphate (ATP), or a pharmaceutically acceptable salt thereof.

According to a preferred embodiment, the compound of formula (I) is adenosine 5′-monophosphate (AMP):

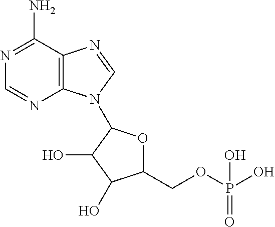 [SMILES: Nc1ncnc2c1ncn2C1OC(COP(=O)(O)O)C(O)C1O]

AMP has been shown to have a very good stabilizing effect on liquid preparations of intact antibodies, in particular intact monoclonal antibodies, such as for example bevacizumab.

AMP has been shown to significantly reduce the propensity of intact antibodies, such as, for example, the intact monoclonal antibody bevacizumab, to form aggregates in liquid formulations. Further, AMP has been shown to induce significant reversion, or breaking, of already formed aggregates of intact antibodies, such as for example bevacizumab, into an essentially monomeric state.

For example, addition of AMP to a liquid formulation of intact monoclonal antibody, such as bevacizumab, containing already formed antibody aggregates has been shown to provide a decrease in the amount of aggregates in the liquid formulation, and an increase in the amount of antibody monomers in the liquid formulation, for instance an increase in the proportion of the antibody present in the monomer form of generally from 10% to 30%, may be observed.

Advantageously, aqueous formulations of intact antibody according to the invention comprising AMP may contain less than 20%, even less than 15%, even less than 10% of the antibody in aggregate form, as determined by MALS coupled to AFFF, on storage at a temperature of 40° C. for 35 days.

According to one embodiment, the invention provides a formulation according to the invention comprising the intact monoclonal antibody bevacizumab and AMP, as the compound of formula (I), wherein less than 10% of bevacizumab forms an aggregate as determined by MALS coupled to AFFF during storage at a temperature of 40° C. for 35 days. The invention further encompasses any tautomers of AMP according to the invention.

Particular advantages of AMP include that AMP is widely commercially available, and at a low cost. AMP is a widely used and accepted food additive. AMP is approved by the FDA under GRAS (Generally Recognized As Safe) notification GRN No. 144. AMP is widely used as a flavour enhancer and/or flavour modifier, for example in chewing gum, coffee, tea, sugar substitutes, snack foods, soups and soup mixes.

The use of a non-therapeutic compound, e.g. a known excipient or additive compound, such as AMP as stabilizing agent for liquid formulations of intact antibody presents also further advantages with respect to avoiding potential problems of combinations of the antibody with another therapeutically or physiologically active agent as stabilizer, such as problems of reduced antibody activity or even possible undesired side effects or toxicological effects related to the active agent combination.

According to another embodiment, the compound of formula (I) is adenosine 5′-triphosphate (ATP):

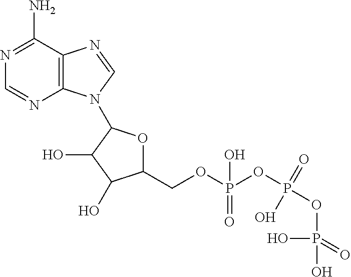 [SMILES: Nc1ncnc2c1ncn2C1OC(COP(=O)(O)OP(=O)(O)OP(=O)(O)O)C(O)C1O]

According to another embodiment, the compound of formula (I) is guanosine 5′-monophosphate (GMP):

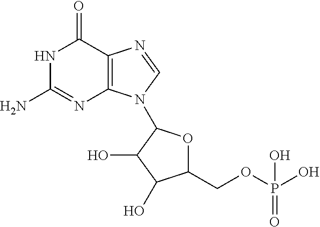 [SMILES: Nc1nc2c(ncn2C2OC(COP(=O)(O)O)C(O)C2O)c(=O)[nH]1]

The formulations of the invention comprise at least one intact antibody. Generally the formulation of the invention will contain one type of intact antibody, in a native form or in a form conjugated to an accessory molecule. However, the formulations of the invention may comprise more than one intact antibody, e.g. two or three different intact antibodies.

The intact antibody according to the invention is preferably an intact monoclonal antibody. The intact monoclonal antibody may be an immunoglobulin, for example particularly an IgG1, IgG2, IgG2b, IgG3 or IgG4. The intact monoclonal antibody may be any known therapeutic, diagnostic or preventative intact monoclonal antibody drug, such as, for example, Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab, or Ustekinumab. Intact monoclonal antibodies of particular interest include IgG1, IgG4 and monoclonal antibodies having an Fc region substantially similar to that of IgG1, including, for example, Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab, or Ustekinumab.

According to a preferred embodiment, there is provided a stable antibody formulation according to the invention wherein the intact antibody is bevacizumab.

Based on findings of the inventors, it is considered that the efficacy of compounds of formula (I) for reducing the propensity of intact antibodies to form aggregates, and for inducing reversion of already formed aggregates of intact antibody molecules to an essentially monomeric state, is due to interference of the compounds of formula (I) with an aggregation specific binding site on the Fc region of the antibody molecule, thereby inhibiting, or blocking, a second antibody molecule from effectively binding to the aggregation binding site on a first antibody molecule. This inhibits the formation of aggregates between the antibody molecules, due to a mechanism of competitive binding at the aggregation binding site on the first antibody molecule.

Suitable liquid carriers for the antibody formulation according to the invention include, for example, water, ethanol, polyols e.g. glycerol, propolyene glycol, polyethylene glycol, vegetable oils, etc. Aqueous carriers may be preferred. Preferred pharmaceutically acceptable carriers include sterile aqueous solutions or dispersions, particularly sterile injectable solutions or dispersions. Injectable solutions or dispersions may typically be based upon injectable sterile saline or phosphate-buffered saline (PBS) or other injectable carriers known in the art.

Aqueous formulations according to the invention may generally have a pH in the range of pH 4.0 to pH 8.0, for example a physiological pH, for example a pH around pH 7.0. According to the invention there is provided a formulation according to the invention wherein the formulation is a pharmaceutical formulation, notably formulated for administration in a mammal, typically a human.

Pharmaceutical formulations according to the invention may additionally contain pharmaceutically acceptable buffers (e.g. PBS buffer). Pharmaceutical formulations according to the invention may additionally contain pharmaceutically acceptable excipients, such as for example known pharmaceutically acceptable preservatives, antibacterial agents, dispersing agents, suspending agents, wetting agents, emulsifying agents, flavouring agents, colouring agents, etc. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia.

The desired concentration of intact antibody in the formulation according to the invention, will depend, amongst others, on the particular antibody used, the pathology to be treated, the dosage form, the dosage regime, the patient to be treated, etc. In general, in aqueous formulations of an antibody for parenteral administration (e.g. by injection or infusion) concentration of an antibody in the range from about 1 mg/ml to about 25 mg/ml, e.g. from about 2 mg/ml to about 20 mg/ml, are usual. According to one embodiment, the invention provides a formulation according to the invention wherein bevacizumab is at a concentration in the range from about 1 mg/ml to about 25 mg/ml, preferably from about 2 mg/ml to about 20 mg/ml.

The desired concentration of compound of formula (I) in the formulation according to the invention will depend, amongst others, on the concentration of the antibody in the formulation, the extent of stabilization desired, etc. For instance in an aqueous formulation of antibody according to the invention for parenteral administration (e.g. by injection or infusion), a concentration of compound of formula (I) in the range from about 0.001 mg/ml to about 50 mg/ml, e.g. from about 0.01 to about 20 mg/ml, may be envisaged. According to one embodiment, the invention provides a formulation according to the invention wherein AMP is at a concentration in the range from about 0.1 mg/ml to about 10 mg/ml.

Generally, the molar ratio of the compound of formula (I) to the intact antibody is in the range from about 0.1:1 to about 500:1, preferably from about 1:1 to about 200:1. In a particular embodiment, the molar ratio of the compound of formula (I) to the intact antibody is in the range from about 1:1 to about 100:1, in particular 1:1 to about 50:1 such as for example from about 1:1 to about 10:1.

Formulations of this invention may be administered in any manner including parenterally, transdermally, rectally, transmucosally, intra-ocular or combinations thereof. Parenteral administration includes, but is not limited to, intravenous (i.v.), intraarterial, intraperitoneal, subcutaneous, intramuscular, intrathecal, and intraarticular. The compositions of the invention may also be administered in the form of an implant, which allows a slow release of the compositions as well as a slow controlled i.v. infusion.

According to a preferred embodiment, the invention provides a formulation according to the invention wherein the formulation is a pharmaceutical formulation suitable for injection in human (e.g. intravitreal or intravenous). In a particular embodiment the formulation is a pharmaceutical formulation suitable for ocular injection in humans (e.g. intravitreal). In another embodiment the formulation is a pharmaceutical formulation suitable for intravenous injection in humans.

Formulations of the invention, together with a conventionally employed adjuvant, carrier, diluent or excipient, may be placed into the form of pharmaceutical compositions and unit dosages thereof, and in such form may be employed as liquids such as solutions, suspensions, emulsions, elixirs, or capsules filled with the same, or in the form of sterile injectable solutions. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Injectable compositions are typically based upon injectable sterile saline or phosphate-buffered saline or other injectable carriers known in the art.

The formulations of the present invention may be provided in the form of a kit comprising in one or more container(s) a formulation according to the invention together with instructions for use of said formulation.

The formulation may be adapted for delivery by repeated administration.

Stabilized intact antibodies according to the invention and formulations thereof, obtainable by a process or a method according to the invention, are useful in the prevention and/or treatment of a disease or a disorder such as immunological diseases, autoimmune diseases, infectious diseases, inflammatory diseases, neurological diseases, neovascular diseases, or oncological diseases.

According to one embodiment there is provided a formulation according to the invention for use as a medicament.

In particular, formulations according the invention may be envisaged for the prevention or treatment of a disease or a disorder selected from immunological diseases, autoimmune diseases, infectious diseases, inflammatory diseases, neurological diseases, neovascular diseases, or oncological diseases.

According to a particular embodiment of the invention there is provided a formulation according the invention for the prevention or treatment of a disease or a disorder selected from a cancer, or a neovascular age-related macular degeneration disease (AMD).

According to one embodiment of the invention there is provided a method of preventing, treating or ameliorating a disease or a disorder selected from immunological diseases, autoimmune diseases, infectious diseases, inflammatory diseases, neurological diseases, neovascular diseases, or oncological diseases, said method comprising administering in a patient in need thereof a prophylactic or therapeutically effective amount of a stable intact antibody formulation according to the invention or a formulation of a stabilized intact antibody obtainable by a process or a method according to the invention.

According to a particular embodiment of the invention there is provided a method of preventing, treating or ameliorating a neovascular age-related macular degeneration (AMD) or a disorder associated with AMD, said method comprising administering in a subject in need thereof a prophylactic or therapeutically effective amount of a stable bevacizumab formulation or a formulation of a stabilized bevacizumab obtainable by a process or a method according to the invention.

According to another aspect, the invention provides a method of preventing, treating or ameliorating a cancer, said method comprising administering in a subject in need thereof a prophylactic or therapeutically effective amount of a stabilized antibody formulation or a formulation of a stabilized bevacizumab according to the invention.

Particularly considered cancers include metastatic cancers, e.g. selected from colon or rectal cancer.

Typically, for cancer treatment such as colorectal cancer, the therapeutically effective dose of a stabilized bevacizumab according to the invention is from about 3 mg/kg body weight to about 20 mg/kg body weight.

The dosage administered, as single or multiple dose(s), to an individual will vary depending upon a variety of factors, including pharmacokinetic properties, patient conditions and characteristics (gender, age, body weight, health, and size), extent of symptoms, concurrent treatments, frequency of treatment and the effect desired.

According to another aspect of the invention, there is provided a method of stabilizing an intact antibody in aqueous solution by combining said intact antibody with a compound of formula (I).

According to one embodiment, there is provided a process for the preparation of an intact antibody or a formulation thereof comprising the steps of:

(i) combining said intact antibody with a compound of formula (I) into a liquid mixture or formulating said intact antibody in a liquid medium containing a compound of formula (I);

(ii) collecting the liquid mixture or liquid medium obtained under step (i) containing the stabilized intact antibody wherein the percentage of monomers of intact antibody is increased as compared to intact antibody prepared in absence of the said compound of formula (I).

Typically, the percentage of monomers of stabilized intact antibody is of about at least 90% after 35 days at a temperature of 40° C. at 25 mg/ml.

In a particular embodiment a method is provided according to the invention wherein the said intact antibody is bevacizumab. For example, bevacizumab used in a method or process according to the invention may be obtained by a process as described in Presta et al., 1997, above.

In a particular embodiment there is provided a method or process according to the invention wherein the said compound of formula (I) is AMP, ADP or ATP, particularly AMP.

The method or process according to the invention may also usefully be applied for decreasing the aggregation ability of an intact antibody during its production process and/or in rescuing production batches containing already aggregated antibodies by reverting them into an essentially monomeric state.

The method or process according to the invention may be usefully applied for preparing stable formulations of intact antibodies presenting an increased shelf-life and enabling multiple dosing conditioning.

The invention is further illustrated by the following-non limiting examples.

### EXAMPLES

### General Procedures & Conditions

The following studies are conducted to support the influence of compounds of formula (I), such as AMP, ATP and GMP on the stability of intact antibodies such as bevacizumab and the like. Monomer percentages of the intact antibody are measured to determine whether its association with a compound of formula (I) in a single formulation influences the aggregation state of this antibody protein. Since aggregates have been observed to cause severe side-effects, this study is of great importance for anticipating beneficial effects in clinical use.

The following abbreviations refer respectively to the definitions below: mM (millimolar), nm (nanometers), AFFF (asymmetrical flow field-flow fractionation), MALS (multi-angle light scattering), UV (ultraviolet).

### Example 1

### Comparison of the Stability of Bevacizumab Alone and in Association with Adenosine 5′-monophosphate (AMP)

Four different samples were tested:

Commercial formulation of bevacizumab (Avastin®, Roche Pharma, Reinach, Switzerland) comprising 25 mg/mL bevacizumab in 51 nM phosphate buffer, pH 6.2 containing 60 mg/mL trehalose dehydrate and 0.04% polysorbate 20 was dialyzed overnight into isotonic buffers to reduce excipients present in the commercial product and to change the pH. A 50 mM phosphate buffer pH 7.0 was used. The buffer choice was based on a pH range and buffer capacity that is tolerated physiologically and that is acceptable for the stability of antibodies.

After dialysis, the bevacizumab preparation at a concentration of 25 mg/mL was stored for 7 days at a temperature of 40° C. and pH 7.0 to stress the antibody and induce formation of aggregates.

A first sample of bevacizumab was separated (in order to test aggregation of bevacizumab alone).

Adenosine 5′-monophosphate powder (purity 99%, Acros Organics) was added in three different concentrations to the stressed bevacizumab sample at 25 mg/ml, obtaining the following molar ratios:
i. bevacizumab:AMP 1:153
ii. bevacizumab:AMP 1:15.3
iii. bevacizumab:AMP 1:1.53

All samples were stored at a temperature of 40° C. during 28 days. Samples were analyzed directly after preparation (t₀) and after 7, 14 and 28 days. The aggregation state of the antibodies was measured by multi-angle light scattering (MALS) after separation by asymmetrical flow field-flow fractionation (AFFF). The concentration of bevacizumab was determined by UV spectroscopy at 280 nm, based upon an extinction coefficient of 1.7 cm ml/mg. Data were collected and analyzed with Astra software (Wyatt Technology Europe GmbH, Dernbach, Germany). The aggregation state was expressed as the percentage of monomers versus time.

Further control experiments on the stability of bevacizumab alone were carried out: Concentration effect (5, 10, 18 and 25 mg/ml in 50 mM phosphate buffer pH 6.2) and effect of pH as well as storage temperature (pH 5.0, pH 6.0 and pH 7.0 at 4° C., 25° C. and 40° C. during 28 days) on antibody stability.

The association of bevacizumab with AMP causes a surprising stabilization of the antibody in comparison with the sample of bevacizumab alone (FIG. 1). After 14 days of storage at a temperature of 40° C. at pH 7.0, the percentage of monomers in the formulations of bevacizumab with AMP is higher than 94% (n=3). After 28 days of storage at 40° C. at pH 7.0, the percentage of monomers is still around 90% (n=3) (FIG. 1) for a molar ratio of bevacizumab:AMP=1:153; and is still higher than 80% after 14 days of storage at 40° C. at pH 7.0 and higher than 76% (n=3) after 28 days of storage at 40° C. at pH 7.0 for a molar ratio of bevacizumab:AMP=1:15.3 or 1:1.53. This is compared to average monomer percentages (n=3) of 75% after 14 days of storage at 40° C. at pH 7.0, and 71% after 28 days of storage at 40° C. at pH 7.0 for bevacizumab alone (FIG. 1).

These data clearly show that the combination of an intact antibody such as bevacizumab with a compound of formula (I) such as adenosine 5′-monophosphate (AMP) leads advantageously to stabilized antibody formulations.

### Example 2

### Effect of Adenosine 5′-monophosphate (AMP) on Commercial Formulation of Bevacizumab (Avastin®)

Samples of commercial formulation of bevacizumab (Avastin®, Roche Pharma, Reinach, Switzerland) are combined with AMP at three molar ratios (1:1, 1:10 and 1:100 Avastin®:AMP). All samples are stored at a temperature of 40° C. for 28 days and stability is measured as described in Example 1 and compared to a sample of Avastin® alone stored under the same conditions.

Compared to the sample of the commercial Avastin® formulation alone, a significant stabilization of the antibody (increase in the amount of monomers) is observed for both the 1:10 and 1:100 samples (p<0.05). For the 1:10 sample, a significant stabilization is observed at t₁, t₁₄ and t₂₈, whereas, for the 1:100 sample, such a stabilization is observed only at longer incubation times (t₁₄ and t₂₈) (FIG. 2). Therefore, compared to the other molar ratios 1:1 and 1:100, the 1:10 sample results in a better stabilization of the antibody. In conclusion, those results confirm those of Example 1 and show that a compound according to formula (I) such as adenosine 5′-monophosphate (AMP) is also able to further stabilize an unmodified commercial antibody formulation.

### Example 3

### Comparison of the Stability of Bevacizumab Alone and in Association with Guanosine 5′-monophosphate (GMP) or Adenosine 5′-triphosphate (ATP)

Commercial formulation of bevacizumab (Avastin®, Roche Pharma, Reinach, Switzerland) is pre-stressed after dialysis into PBS at pH 7.0 as described in Example 1 (for 7 days at a temperature of 40° C.). After pre-stressing, Avastin® samples are combined with either ATP or GMP at three Avastin®: compound of formula (I) molar ratios (1:1, 1:10 and 1:100). All samples are stored at a temperature of 40° C. for 28 days and stability is measured as described in Example 1 and compared to a sample of Avastin® alone stored under the same conditions. For GMP, a dilution of GMP was made in PBS pH 7.0 and pH was re-adjusted to pH 7.0 before the combination with Avastin® to prevent the risk of higher order aggregates caused by the addition of NaOH directly to the antibody formulation.

A concentration dependent stabilization of Avastin® is observed after addition of ATP up to 14 days. At t₂₈, no significant difference is observed between the sample of Avastin® alone and the 1:1 and 1:10 combinations. The 1:100 sample shows a significant stabilization of the antibody after 28 days of storage, although a small percentage of higher order aggregates is also observed. These aggregates are probably due to the adjustment of the pH of this sample. A concentration dependent stabilization of Avastin® is also observed after addition of GMP: At all timepoints, the 1:100 formulation is the most effective in aggregation breaking, followed by the 1:10 and thereafter the 1:1 sample. Therefore, at an initial stage (e.g. 1 day of storage at 40° C.: t₁), a stabilizing effect is observed for all three molar ratios (FIG. 3A) after addition of ATP, whereas GMP seems to be less effective as only the 1:100 sample shows an ability to stabilize the antibody, whereas both the 1:1 and 1:10 samples are destabilizing. At later stage (e.g. 28 days of storage at 40° C.: t₂₈), ATP still shows a significant stabilizing effect on the antibody for the 1:100 samples, however the 1:1 and 1:10 samples show a similar stability as the antibody alone (FIG. 3B).

Thus, although ATP shows aggregation breaking effects, these effects are most pronounced directly after addition of the excipient to the antibody. It appears that it takes more time for GMP to interact with the antibody in order to interfere with the formation of antibody dimers.

In conclusion, excipients of formula (I) possess stabilizing properties. Short-term effects on the antibody are most pronounced for ATP, whereas GMP shows the most distinct stabilizing properties after 28 days of storage at 40° C.

### Example 4

### Comparison of the Stability of Antibodies Alone and in Association with a Compound of the Invention

Stabilizing effects of compounds of formula (I) according to the invention on various antibodies are assessed as follows:

Long-Term Stability Studies

The antibody at a concentration of 25 mg/mL in 20 mM histidine buffer pH 6.0 is combined with a compound of formula (I) (such as AMP) from a stock solution in the same buffer, at molar ratios of antibody: compound of 1:1 and 1:10. The resulting samples where the antibody is at a concentration of 20 mg/ml or higher are then stored either at normal storage temperature (5° C.) or at elevated temperatures (e.g. 25° C. or 40° C.). Aggregation state is then measured during storage such as immediately after sample preparation, 2 weeks, 1 month, 3 months and 6 months after starting storage based on the proportions of monomers, dimers and larger antibody aggregates in each samples by various techniques such as Asymmetrical-Flow Field-Flow-Fractionation (AFFF), Size Exclusion Chromatography, or Analytical Ultracentrifugation. Comparison of aggregation state in the presence and in the absence of compounds of formula (I) demonstrates their ability to prevent aggregation.

Short-Term Stability Studies Under Stress Conditions

The antibody 25 mg/mL in 20 mM histidine buffer pH 6.0 is pre-stressed using known aggregating conditions (e.g. temperature, pH, agitation for example as described in Kiese et al., 2008, Journal of Pharmaceutical Sciences, 97(10), 4347-4366) followed by the addition of a compounds of formula (I) such as AMP at molar ratios of Mab:compound of 1:1 and 1:10 in buffer. The resulting samples where the antibody is at a concentration of 20 mg/ml or higher are then analyzed for determining their aggregation status immediately after the addition of compounds of formula (I) and 1 week after starting, based on the proportions of monomers, dimers and larger antibody aggregates in each samples by various techniques such as Asymmetrical-Flow Field-Flow-Fractionation (AFFF), Size Exclusion Chromatography, or Analytical Ultracentrifugation. Comparison of aggregation state in the presence and in the absence of compounds of formula (I) demonstrates their ability to reverse aggregation

1-27. (canceled)

28. A stable antibody formulation comprising a liquid carrier, an intact antibody and a compound of the formula (I): 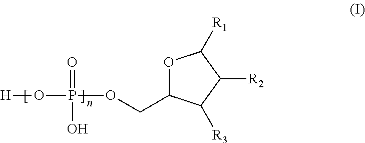
wherein R₁ is a nucleobase selected from the group comprising adenine, guanine, thymine, uracil, xanthine, ethanoadenine, inosine, orotidine, or cytosine; R₂ is H or OR₄ wherein R₄ is H or a C₁₋₄ alkyl group; R₃ is H or OR₅ wherein R₅ is H or a C₁₋₄ alkyl group; and n is an integral from 1-3, or a pharmaceutically acceptable salt or a tautomer thereof.

29. The formulation according to claim 28, wherein R₂ is H or OH and R₃ is H or OH.

30. The formulation according to claim 28, wherein the compound of formula (I) is adenosine 5′-mono, -di, or -triphosphate or a pharmaceutically acceptable salt or a tautomer thereof.

31. The formulation according to claim 28, wherein the compound of formula (I) is adenosine 5′-monophosphate (AMP) or a tautomer thereof.

32. The formulation according to claim 28, wherein the compound is guanosine 5¹-monophosphate (GMP) or a tautomer thereof.

33. The formulation according to claim 28, wherein the intact antibody is conjugated to an accessory molecule or is a native antibody.

34. The formulation according to claim 28, wherein the intact antibody is bevacizumab.

35. The formulation according to claim 28, wherein the formulation is a pharmaceutical formulation.

36. A pharmaceutical unit dosage form suitable for ocular or intravenous administration to a mammal comprising an antibody formulation according to claim 28 in a suitable container.

37. A method of stabilizing an intact antibody in liquid carrier by combining said intact antibody with a compound of formula (I): 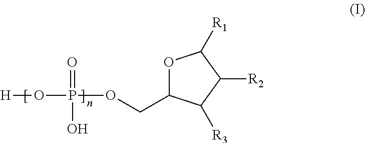
wherein R₁ is a nucleobase selected from the group comprising adenine, guanine, thymine, uracil, xanthine, ethanoadenine, inosine, orotidine, or cytosine; R₂ is H or OR₄ wherein R₄ is H or a C₁₋₄ alkyl group; R₃ is H or OR_{S} wherein R₅ is H or a C₁₋₄ alkyl group; and n is an integer from 1-3, or a pharmaceutically acceptable salt or a tautomer thereof.

38. A stabilized intact antibody or a formulation thereof obtainable by a method according to claim 37.

39. The stabilized intact antibody or a formulation thereof according to claim 38 wherein the said intact antibody is bevacizumab.

40. The stabilized intact antibody or a formulation thereof according to claim 38 wherein the compound of formula (I) is adenosine 5′-monophosphate (AMP) or a pharmaceutically acceptable salt or a tautomer thereof.

41. The stabilized intact antibody or a formulation thereof according to claim 38 wherein the compound of formula (I) is adenosine 5′-triphosphate (ATP) or a pharmaceutically acceptable salt or a tautomer thereof.

42. The stabilized intact antibody or a formulation thereof according to claim 38 wherein the compound of formula (I) is guanosine 5′-monophosphate (GMP) or a pharmaceutically acceptable salt or a tautomer thereof.

43. A pharmaceutical formulation comprising a stabilized antibody or a formulation thereof, according to claim 38.

44. A method of preventing, treating or ameliorating a disease or a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD), said method comprising administering to a subject in need thereof a prophylactic or therapeutically effective amount of a stable bevacizumab formulation according to claim 28 or a pharmaceutical formulation according to claim 43.
